(19) United States
(12) Patent Application Publication
(10) Pub. No.: US 2007/0196839 A1
(43) Pub. Date: Aug. 23, 2007

(54) CONSIDERATIONS, EVALUATIONS, INVESTIGATIONS AND SEARCHING

(75) Inventors: Richard Pinchin, Birmingham (GB); John Buckleton, Auckland (NZ)

Correspondence Address: MERCHANT & GOULD PC, P.O. BOX 2903, MINNEAPOLIS, MN, 55402-0903, US

(73) Assignee: The Secretary of State of the Home Department, Birmingham (GB)

(21) Appl. No.: 11/617,268

(22) Filed: Dec. 28, 2006

Related U.S. Application Data
(63) Continuation of application No. 10/450,597, filed on Jan. 20, 2004, now abandoned.

(30) Foreign Application Priority Data
Mar. 28, 2002 (GB) 0207365.8

(51) Int. Cl. C12Q 1/68; G06F 19/00

(52) U.S. Cl. 435/6; 702/20

(57) ABSTRACT

Methods of considering DNA based links between two or more situations are provided. Amongst the methods is a method including obtaining a plurality of test results, each test result relating to a situation, each test result including information on the DNA from that situation, the plurality of test results providing a group of test results; selecting a plurality of test results from the group of test results, the plurality of results for the combination of test results; considering a genotype as possibly giving rise to each of the test results of the combination, evaluating the support for that genotype giving rise to all of the test results of the combination; considering the genotype as a DNA based link between the situations for the test results in a combination if the support meets the defined criteria. The methods provide new techniques for considering preexisting DNA test results and/or new DNA test results so as to establish links between them in terms of the genotypes which are supported as contributing to them. The methods enable the evaluation of the support for genotypes given the test results in relation to various combinations of test results.

This invention concerns improvements in and relating to considerations, evaluations and investigations, particularly but not exclusively in relation to highlighting genotypes of interest, and to searching, particularly but not exclusively in relation to searching of databases of genotypes.

Substantial numbers of DNA test results exist from various scenes, samples taken from scenes and replicates of samples. These relate to both solved and unsolved incidents. It is desirable to be able to obtain as much information as possible from the consideration or evaluation of these test results. The present invention seeks to provide a significantly more powerful tool for this purpose than presently exists.

A substantial number of genotypes are also recorded, for instance in The National DNA Database (UK Registered Trade Mark) of genotypes in the UK, and these provide a potential source of information to be considered. The present invention seeks to provide a significantly more powerful tool for considering the information in such databases.

The present invention has amongst its aims to provide new techniques which enable useful information to be obtained by considering pre-existing DNA test results alone or in combination with new DNA test results so as to establish links between some of those test results in terms of genotypes which are supported as contributing to them. The present invention has amongst its aims to evaluate the more supported genotypes given the test results in relation to various combinations of test results.

According to a first aspect of the invention we provide a method of considering DNA based links between two or more situations, the method including:

obtaining a plurality of test results, each test result relating to a situation, each test result including information on the DNA from that situation, the plurality of test results providing a group of test results;

selecting a plurality of test results from the group of test results, the plurality of results forming a combination of test results;

considering a genotype as possibly giving rise to each of the test results of the combination, evaluating the support for that genotype giving rise to all of the test results of the combination;

considering the genotype as a DNA based link between the situations for the test results in the combination if the support meets defined criteria.

According to a second aspect of the invention we provide a method of considering DNA based links between two or more situations, the method including:

obtaining a plurality of test results, each test result relating to a situation, each test result including information on the DNA from that situation, the plurality of test results providing a group of test results;

selecting a plurality of test results from the group of test results, the plurality of results forming a combination of test results;

considering a genotype as possibly giving rise to each of the test results of the combination, evaluating the support for that genotype giving rise to all of the test result of the combination;

considering the genotype as a DNA based link between the situations for the test results in the combination if the support meets defined criteria;

comparing a genotype which is considered a DNA based link against records of genotypes to identify matching genotypes in the records.

The second and/or third aspects of the invention may include any of the features, options or possibilities set out elsewhere in this document, including the third and fourth aspects of the invention.

The situations may be test results for different scenes and/or different samples from the same or different scenes and/or replicates from the same samples or from different samples. The situations may include test results for one or more known individuals. The test results may relate to mixtures and/or single contributor cases. The test results may relate to complete and/or partial profiles.

The information on the DNA may be the allele present at a loci, ideally for at least 6 different loci. The information may include the peak height and/or peak area for those alleles. Complete or partial profiles may be accepted as test results for application of the method.

The group of test results may be stored as a group or may be pulled together from discrete sources. The discrete sources may be pulled together to form a group. Alternatively one or more test results from such sources may be pulled together to form a group for the purposes of the application of the method.

All possible combinations of test results for the test results in the group may be considered. Pairs, triplets and quadruplets of test results may particularly be considered.

The evaluation a direct evaluation of the support for a genotype giving the combination of test results or may involve an evaluation of the support for a given genotype giving each of the test results, the individual evaluations being combined to give the overall evaluation.

The support may meet the defined criteria when the probability that the genotype could have given rise to the test results of the combination is above a given level. The level may be predetermined.

The support may meet the defined criteria when an expression of the support that the genotype could have given rise to the test results of the combination is below a given level. That level may be predetermined.

The method may be used to establish DNA based links between different scenes. The method may be used to establish DNA based links between different samples taken from different parts of the same scene. The method may be used to establish DNA based links between replicates of the same sample. The method may be used to establish DNA based links between combinations of such situations. The method may be used to establish a DNA based link between a person whose genotype is known and one or more situations.

The method may be used to establish DNA based links between test results which are ambiguous or which could be suggested to be ambiguous. Such test results might be one or more of test results which are or could be mixtures and/or test results which are or could involve low levels of DNA in the sample for one or more persons (low levels could be though of as less than 500 pg or even less than 100 pg of DNA) and/or test results which do or could involve effects due to stutter and/or allele drop out and/or allele contamination and/or preferential amplification.

The method may be used to establish a genotype or genotypes which are DNA based links between situations and which are then matched to an existing genotype record, ideally for a known person.

According to a third aspect of the invention we provide a method of considering DNA based links between two or more situations, the method including:
obtaining a plurality of test results, each test result relating to a situation, the test results including information on the DNA from the corresponding situation, the plurality of test results providing a group of test results;
selecting a combination of test results which includes a plurality of test results from the group of test results;
considering one or more genotypes as possibly giving rise to the combination of test results and evaluating for each of those genotypes the support that the genotype gave rise to the combination of test results;
considering a genotype whose support meets defined criteria as a DNA based link between the situations of the combination.

The third aspect of the invention may include any of the features, options or possibilities set out in this document, but particularly from amongst the following.

Preferably the evaluation of the support for a genotype giving rise to the combination of test results includes a consideration of the probability of the test results arising given that genotype and the probability of occurrence of that genotype. Preferably the evaluation of the support for the genotype giving rise to the combination of test results includes a consideration of the overall probability of the test results arising given that genotype and the probability of occurrence of that genotype, for all possible genotypes. Preferably the evaluation of the support for a genotype giving rise to the combination of test results includes a consideration of the probability of the test results arising given that genotype and the probability of occurrence of that genotype against a consideration of the overall probability of the test results arising given that genotype and the probability of occurrence of that genotype, for all possible genotypes.

Preferably the evaluation of the support for a genotype giving rise to the combination of test results is defined by

$$Pr ( Gr \vert D… ) = \frac{{\prod }_{\text{?}} Pr ( D \text{?} \vert Gr )}{{\sum }_{i} {\prod }_{\text{?}} Pr ( D \text{?} \vert Gr ) Pr ( Gr )} for all 1. \\ \text{?} \text{indicates text missing or illegible when filed}$$

where Gₗ represents the particular genotype, D represents the combination of test results, potentially including test results due to various scenes and/or samples from scenes and/or replicates of samples from scenes, i represents the range of replicates, j the range of samples, k the range of scenes and l the range of genotypes under consideration.

Preferably the method is applied to a plurality of combinations selected from the group of test results. Each possible combination of test results from amongst the group of test results may be considered. Combinations may include two, three, four or more test results from the group. A combination may include one or more test results for a situation which is from a different scene to one or more other situations represented by test results in the group. A combination may include one or more test results for a situation which is from a different sample to one or more other situations represented by test results in the group. A combination may include one or more test results for a situation which is from a different replicate to one or more other situations represented by test results in the group.

Each possible genotype may be considered as giving rise to the combination of test results. One or more limits may be applied to the genotypes which are considered from amongst the full set of possible genotypes. The limits may be based on one or more rules as to genotypes which could not practically give one or more of the results in the combination being considered.

The evaluation may be expressed as a posterior probability.

Preferably the evaluation of the support for a genotype giving rise to the group of test results includes a consideration of the effect of one or more of contamination of the test results and/or allele drop out from the results and/or stutter in the results and/or preferential amplification of the results.

The method may be used to consider one or more test results which are mixtures. The method may be used to consider one or more test results which contain low levels of DNA from one or more persons. The method may be used to consider one or more test results where there is ambiguity or suggested ambiguity as to the contributors and/or the genotypes of the contributors. The method may be used to consider one or more test results to which there is only one contributor and/or for which the genotype is known.

A DNA based link may be present where the support that a genotype could give rise to the test result meets criteria for each of the test results for which a link is determined. The criteria may be a predetermined support level. The defined criteria may be a genotype whose support for giving rise to the test results in the combination is above a defined level. The level may be predefined.

A DNA based link may be used to suggest or confirm a link between one or more situations. The link may support other links for those situations based on other evidence types. The link may suggest links for which links had not previously been suggested. The link may be used to direct subsequent investigation of the situations and/or events which gave rise to those situations and/or individuals behind those situations by law enforcement agencies. The DNA based link may be used as evidence in legal proceedings.

A genotype which is considered as a DNA based link between the situations of the combination may be used in a further consideration. The further consideration may include the review of possible matches between the genotype and a collection of genotype records. The existence of a match may be deemed to occur where correspondence at or above a given level of correspondence occurs. The given level may be at least 80%, and ideally at least 90%, of alleles in common between the genotypes. The recorded genotypes may be genotypes of known individuals. The further consideration may link the genotype to an individual. The further consideration may link the situations of the combination of test results to an individual.

The test result may be obtained, or have been obtained, by PCR based amplification of DNA collected from the situation. The test result may be obtained, or have been obtained, by establishing allele identities for one or more loci of the DNA. The peak area and/or peak height for the alleles may be obtained. The test results may be obtained for use in the method and/or may have previously been obtained for other purposes. The test results may be reused in the method after use in other analysis and/or consideration methods.

The situations may be test results for different scenes and/or different samples from the same or different scenes and/or replicates from the same samples or from different samples. The situations may include test results for one or more known individuals. The test results may relate to mixtures and/or single contributor cases. The test results may relate to complete and/or partial profiles.

The number of test results obtained may be more than 10, more than 100 or even more than 1000. The information on the DNA may be the identity of one or more alleles at one or more loci. The identity and peak height and/or peak area may be obtained.

The group of test results may be in a formal group or may be test results stored in a variety of locations. The group of test results may include one or more test results from the test results for an investigation and/or one or more test results from the test results for another, (potentially at the time of combinations selection, unrelated) investigation and/or one or more test results from a centralised store, such as The National DNA Database. The group of test results may include all available test results.

According to a fourth aspect of the invention we provide a method of considering DNA based links between two or more situations, the method including:
obtaining a first test result for a first situation, the first test result including information on the DNA from the first situation;
considering a genotype as possibly giving rise to the first test result and evaluating the support that the genotype gave rise to the first test result, repeating the evaluation of the support for a plurality of other genotypes, generating a set of possible genotypes based on the evaluation with respect to the first test result;
obtaining a second test result for a second situation, the second test result including information on the DNA from the second situation;
considering a genotype as possibly giving rise to the second test result and evaluating the support that this genotype gave rise to the second test result, repeating the evaluation of the support for a plurality of other genotypes, generating a set of possible genotypes based on the evaluation with respect to the second test result;
combining the set of possible genotypes for the first test result and the set of possible genotypes for the second test result, genotypes present in the first set and the second set being given a higher ranking in the combined set than genotypes not present in one or more of the sets;
considering one or more of the higher ranked genotypes in the combined set as a DNA based link between the first situation and the second situation.

The fourth aspect of the invention may include any of the features, options or possibilities set out elsewhere in this document.

Each possible genotype may be considered as giving rise to a test result. One or more limits may be applied to the genotypes which are considered from amongst the full set of possible genotypes. The limits may be based on one or more rules as to genotypes which could not practically give the result being considered. The same or different genotypes may be considered for the different test results. Different, but preferably the same rules may be used as limits in considering each test result.

The evaluation of the support may involve a determination of the mixture proportions contributed by different individuals. The determination may involve the comparison of the observed and expected peak height and/or peak area results at one or more loci. The peak area expected may be subtracted from the peak area observed for a locus, squared, and then summed with the values for the other loci to give a residual. Account may be taken of errors in mixture proportion and/or peak area determinations.

The evaluation of the support may be used to rank the set. Preferably the evaluation of the support includes a least squares based evaluation. The lower the value of the residual a genotype has, the higher ranking it may be given in the ranked evaluation. Preferably the same evaluation is used for each genotype considered and/or for each test result considered.

The evaluation may produce a list of possible genotypes for the first and second test results. The list may be ranked. The set may be in the form of a list.

The set or ranked evaluation or evaluations may include a pre-determined number of genotypes. The number may be at least 200 or even at least 400. The number may be less than 1000. The set or ranked evaluation may include all genotypes with a support above a pre-determined threshold.

Preferably the genotypes included in a set or ranked evaluation are ranked within that evaluation.

Preferably the combining of the set for the first test result and the set for the second test result includes, for genotypes present in the first set and in the second set, adding the support for that genotype for the first set to the support for that genotype for the second set. The residual value for a genotype in the first set may be added to the residual value for the same genotype in the second set. Preferably the combining of the set or ranked evaluation for the first test result and the set or ranked evaluation for the second test result includes combining the support for that genotype for the set or ranked evaluation it is present in with a dummy support for the set or ranked evaluation it is absent from. The dummy may be a pre-set support value. The dummy may be a multiple of the support of the least likely genotype in the set or ranked evaluation from which the genotype was absent. The multiple is preferably greater than 1, such as 2.

Preferably the genotypes are ranked within the combined set or combined evaluation. Preferably genotypes present in each of the sets or ranked evaluations receive a high ranking in the combined set or ranked evaluation. Preferably genotypes absent from one or more of the sets of ranked evaluations receive a low ranking in the combined set or ranked evaluation.

The first and second test results may be from situations which differ in terms of the scene and/or the sample and/or the replicate in question.

The method may be applied to three, four or more test results.

The method may be applied to test results in existing records. Each pair of test results and/or triple of test results and/or quadruple of test results and/or higher combinations could be considered according to the method.

The method may be used to consider one or more test results which are mixtures. The method may be used to consider one or more test results which contain low levels of DNA from one or more persons. The method may be used to consider one or more test results where there is ambiguity or suggested ambiguity as to the contributors and/or the genotypes of the contributors. The method may be used to consider one or more test results to which there is only one contributor and/or for which the genotype is known.

A genotype which is considered as a DNA based link between the situations may be used in a further consideration. The further consideration may include the review of possible matches between the genotype and a collection of genotype records. The existence of a match may be deemed to occur where correspondence at or above a given level of correspondence occurs. The given level may be at least 80%, and ideally at least 90%, of alleles in common between the genotypes. The recorded genotypes may be genotypes of known individuals. The further consideration may link the genotype to an individual. The further consideration may link the situations of the combination of test results to an individual.

The test results may be obtained, or have been obtained, by PCR based amplification of DNA collected from the situations. The test results may be obtained, or have been obtained, by establishing allele identities for one or more loci of the DNA. The peak area and/or peak height for the alleles may be obtained. The test results may be obtained for use in the method and/or may have previously been obtained for other purposes. The test results may be reused in the method after use in other analysis and/or consideration methods.

The situations may be test results for different scenes and/or different samples from the same or different scenes and/or replicates from the same samples or from different samples. The situations may include test results for one or more known individuals. The test results may relate to mixtures and/or single contributor cases. The test results may relate to complete and/or partial profiles.

The number of test results considered may be more than 10, more than 100 or even more than 1000.

The number of test results for which sets of possible genotypes are combined may be two, three, four, five or more.

A DNA based link may be used to suggest or confirm a link between one or more situations. The link may support other links for those situations based on other evidence types. The link may suggest links for which links had not previously been suggested. The link may be used to direct subsequent investigation of the situations and/or events which gave rise to those situations and/or individuals behind those situations by law enforcement agencies. The DNA based link may be used as evidence in legal proceedings.

The test results may be selected from a formal group of results or may be test results stored in a variety of locations. The test results may include one or more test results from the test results for an investigation and/or one or more test results from the test results for another, (potentially at the time of combinations selection, unrelated) investigation and/or one or more test results from a centralised store, such as The National DNA Database. The method may be applied to all available test results.

Various embodiments of the invention will now be described, by way of example only.

The present invention is aimed at establishing links between DNA test results obtained from a series of situations. In general the DNA will be collected and analysed using conventional techniques, such as PCR based amplification and gel electrophoresis to identify the alleles occurring at various loci for the DNA.

The situations being considered could be multiple scenes from which DNA is collected and/or multiple samples of DNA from different parts of a single scene and/or even multiple replicates of DNA from a single sample which are analysed and generate test results. The DNA test results considered could, in one or more cases, have arisen from samples taken from known persons in controlled circumstances, such as the genotype profiles stored on The National DNA Database (Registered Trade Mark). The aim is to establish whether there are any well supported genotypes (those offering a sufficiently high probability), given the various separate test results obtained, for a particular combination of situations.

Taking the example of a serial offender there may be scenes which include DNA of that offender and scenes which do not, samples from a given scene which include DNA of that offender and samples which do not and even replicates of a sample which include a report of the DNA of that offender and other replicates which do not, despite those replicates arising from the same sample. Furthermore some of the samples may be single contributor samples and others may be mixtures involving DNA contributed from a plurality of individuals. Determining links between such scenes and/or samples and/or replicates is not an easy task on an effective timescale.

The present invention provides an intelligence tool in which a DNA test result from a situation is considered in combination with one or more test results from other situations. The tool seeks to establish genotypes which are more supported to arise from the test results in that particular combination of such situations and thereby provide a DNA based link between those situations. Of course the tool can be used to establish that there is no likely link between the test results in that combination and still provide some useful information. The combination of test results considered may particularly include test samples from two or more different scenes, but is useful even where the test results are from different samples at the same scene, and even for different replicates of the same sample. The tool can also consider test result combinations which include test results from different types of situation.

The technique can be used to compare test results having one or more different timings, including: the test results from the analysis of present situations, for instance, recently occurred events under active law enforcement agency consideration for which test results are newly available; the test results from the analysis of past situations, for instance, past events no longer under active consideration for which test results were generated at the time or have now been generated; the test results from analysis of known situations, for instance test results obtained from known persons under controlled circumstances (such as those used to generate The National DNA Database.

The tool may be used to investigate speculated or informed links between situations, which links are arrived at through other processes, by indicating a high level of support for one or more genotypes linking those situations given the test results for them. The tool may be used to investigate in a non-premeditated manner a body of test results from situations with a view to generating suggested links. The tool may thus generated suggested links between situations not previously considered in conjunction with one another. The tool can also be used to suggest links between one or more situations associated with a crime or scene and a stored test result, previously obtained from a known individual.

Two main embodiments of the invention are now described with similar intents behind the process they facilitate and the uses they can be put to. In each case the aim is to identify one or more genotypes which is supported given the particular combination of test results (referred to as a partition) and hence the situations behind them. This provides information on links between crimes, locations and the like. As a further part of the process the genotypes identified in this way can then be compared with the genotype for a known situation to identify any matches between that genotype and one of the particular supported genotypes. A link between the set of scenes and a known individual can thus be obtained.

In a first technique there is a general consideration of the probability of a genotype given the combination of test results/partition, using a consideration of the probability of the test results arising given a specific genotype (from amongst the many possibilities for the genotype) and the probability of occurrence of that specific genotype, compared with a consideration of the overall probability of the test results arising given a specific genotype and the probability of that genotype, for all the possible genotypes. The consideration can be represented as

$$Pr ( Gr \vert D… ) = \frac{{\prod }_{\text{?}} Pr ( D \text{?} \vert Gr )}{{\sum }_{i} {\prod }_{\text{?}} Pr ( D \text{?} \vert Gr ) Pr ( Gr )} for all 1. \\ \text{?} \text{indicates text missing or illegible when filed}$$

where i represents the range of replicates, j the range of samples, k the range of scenes and l the range of genotypes under consideration.

This general consideration can be applied to one or more combinations/partitions of test results from amongst the test results available. Indeed all such combinations/partitions can be considered in obtaining suggestions as to those which are linked and/or the level of support for the linking supported genotype. A combination of situations or a partition is a pair, triplet, quadruplet or higher number of test results each from a different situation (such as replicates and/or samples and/or scenes). By considering each of the possible combinations/partitions an indication will be provided of those combinations/partitions which are linked by DNA reported in the test result for each of the situations behind it, due to the high posterior probability obtained from the consideration. A very large number of the combinations/partitions will of course not be linked.

The outcome is one or more supported genotypes, each of the supported genotypes being the link for one or more of the combinations/partitions. Thus a supported genotype X may be suggested as being involved in a partition consisting of test results from five particular scenes; a separate but supported genotype Y may be suggested as involved in four scenes and so on. This linking of situations, particularly between scenes can be of great use in its own right. For instance, it may allow other evidence from a number of scenes to be considered in combination when previously there was no such suggestion of a link. The other evidence in combination may lead to the solving of the crime.

Once the supported genotypes with a high posterior probability given the test results for that combination of situations or partition are obtained these supported genotypes can be used in further considerations. For instance each of those supported genotypes can be considered against records of genotypes for matches or near matches (90%+ of the alleles in common, for example) so as to link the supported genotype to other situations, for instance scenes, samples or replicates. This information can be used to confirm other evidence and/or to direct future enquiries and/or to open up new lines of enquiry too. In particular a link to an individual may be produced.

As a substantial number of the possible genotypes cannot arise given the test results, the consideration is limited down quite significantly from having to consider all the approximately 10²¹ genotypes possible in total. With the processing side performed by a computer operating the defined consideration, the large amount of processing involved can be realistically performed.

The manner in which the probability of the specific genotype given the test results is considered can be as simply or as sophisticatedly considered as is desired. Obviously more sophisticated considerations can give greater confidence and worth to any combinations of situations or partitions for which links are suggested. Thus it is desirable to include within that probability consideration one or more functions or models which can account for one or more factors such as genotype dropout, Pr(G_{D}), allele dropout, Pr(A_{D}), stutter, Pr(S), preferential amplification, Pr(PA) and others. These issues can all have an effect on the test result from a situation compared with the actual genotype behind the DNA in that situation. Accounting for them makes the consideration of the extent of support for the supported genotype more robust.

To this end models for one or more of these factors can be included. An illustration of the way in which these factors can be modelled is provided in “An investigation of the rigor of interpretation rules for STRs derived from less than 100 pg of DNA.” Gill et al., Forensic Science International 112 (2000) 17-40. Models to account for laboratory introduced contamination, allele drop out and stutter in particular are provided. The paper is concerned with accounting for such factors in the analysis of a single test result, however, and is not concerned with the between result considerations involved in this invention. The models are none the less useful in assisting. Other models can be used, however, and other factors can be modelled.

In a second technique a test result for a situation is obtained and is submitted to an evaluation of the likelihood of a genotype arising given that test result. A heuristic approach is taken; the general aim being to list more supported genotypes ranked by residual of Euclidean distance.

The evaluation may use a technique such the Pendulum™ technique detailed in Gill et al, Forensic Science International 91 (1998) 41-53 to generate the starting information. That paper describes the consideration of the relative contribution of different individuals to a mixture of DNA, followed by a consideration of the likelihood of the possible genotypes as having been behind the test result obtained. The contents of that paper and in particular the disclosure of the manner in which the proportions of a mixture and the ranking of likelihood is performed is incorporated herein by reference. The technique involves, for a single test result, the consideration of a number of loci simultaneously when establishing the likely mixing proportions and the likely genotypes, but is only concerned with the analysis of an individual situation. No between situation/test result consideration is involved.

Other techniques for detailing likely genotypes behind an individual test result can be used in a similar way to provide the starting information.

In the preferred embodiment, the starting information is provided by the Pendulum™ technique and the output of this evaluation is a list of genotypes which give the lowest residual value from the evaluation used. In effect the most supported genotypes are assumed to be those with the lowest residuals. These are ranked from the lowest residual up to a cut off point, which could be a residual level, but is normally a number of genotypes (frequently 500). An output listing for the test result of a situation A is provided below in Table A; the Genotype designation letters and residual values are schematic illustrations only.

TABLE A

| Number in list | Genotype | Residual value |
|---|---|---|
| 1 | Genotype AA | 1.002 × 10⁶ |
| 2 | Genotype GR | 1.058 × 10⁶ |
| 3 | Genotype LD | 1.078 × 10⁶ |
| 4 | Genotype HU | 1.092 × 10⁶ |
| . . . | . . . | . . . |
| 499 | Genotype KW | 2.002 × 10⁶ |
| 500 | Genotype LM | 2.097 × 10⁶ |

By repeating the evaluation for another test result, test result B, a further output listing is obtained, as set out in Table B. This output listing is the test result from another situation, such as a sample obtained from another scene, a sample taken from a different part of the same scene or the like.

TABLE B

| Number in list | Genotype | Residual value |
|---|---|---|
| 1 | Genotype JE | 1.011 × 10⁶ |
| 2 | Genotype QP | 1.023 × 10⁶ |
| 3 | Genotype LW | 1.085 × 10⁶ |
| 4 | Genotype AA | 1.101 × 10⁶ |
| . . . | . . . | . . . |
| 499 | Genotype AS | 1,989 × 10⁶ |
| 500 | Genotype YR | 2.001 × 10⁶ |

If the two output listings are then added to one another in a prescribed manner a combined output listing can be obtained. This listing then provides indications as to those, if any, genotypes which are possibly involved in the situations behind both the test results.

The same concept applies even if a combination of more than two situations is being considered.

In this embodiment the prescribed manner of adding the output listings to give the combined output listing involves the following rules. Where the same genotype is present in each of the output listings considered then the residuals for that genotype in each of the output listings are added together. Where a genotype is absent from an output listing, a dummy residual for each output listing the genotype is absent from is provided and the residuals for the output listings the genotypes is present in are added to that dummy residual. The dummy residual in this embodiment, in each case, is the largest residual of that output listing the genotype is absent from multiplied by a factor (two in the illustrated example). The genotype can alternatively by rejected entirely. The combined output listing presents the genotypes in order based on the residual level they have.

Considering the output listings of tables A and B it turns out that Genotype AA is present in both output listings, as is a Genotype DS which had position 206 in Table A and position 56 in Table B. None of the other genotypes were present in both output listings. The combined output listings is represented in Table C.

TABLE C

| Number in list | Genotype | Combined residual value |
|---|---|---|
| 1 | Genotype AA | 2.103 × 10⁶ |
| 2 | Genotype DS | 3.103 × 10⁶ |
| 3 | Genotype GR | 5.060 × 10⁶ |
| 4 | Genotype LD | 5.080 × 10⁶ |
| 5 | Genotype HU | 5.094 × 10⁶ |
| 6 | Genotype JE | 5.205 × 10⁶ |
| 7 | Genotype QP | 5.217 × 10⁶ |
| 8 | Genotype LW | 5.279 × 10⁶ |
| . . . | . . . | . . . |
| 995 | Genotype KW | 6.004 × 10⁶ |
| 996 | Genotype LM | 6.099 × 10⁶ |
| 997 | Genotype AS | 6.183 × 10⁶ |
| 998 | Genotype YR | 6.195 × 10⁶ |

As can be seen the two supported genotypes which are considered possibilities in each of the two output listings stand out in the combined output listing when compared with the other genotypes and their residual values. This accurately reflects the status of these supported genotypes as being more supported candidates from both output listings and the test results behind them, given the respective test results. Genotype AA ranks higher than Genotype DS in view of its higher ranking in each of the output listings.

Whilst the technique is illustrated above in relation to the combining of two output listings, which in turn represent two test results, the technique can be applied to a larger number of output listings and their underlying test results in the same manner.

In practice it is envisaged that the technique, in any of its embodiments, could be applied to a database of existing test results. Each pair of test results, each triplet of test results, each quadruplet of test results and so on could be considered; and as a result the situations behind them. In this way any links between situations can be considered and highlighted in the combined output listing, without speculating on links or suggesting links for consideration first. This is useful in approaching a body of unsolved or unlinked situations with a view to generating fresh avenues for investigation.

The technique could also be employed when a new test result or series of test results are obtained from a new situation, such as a new scene. These test results can be considered in the same way in all possible combinations of situations, or partitions, with the pre-existing test results to see whether this situation is linked to another.

The technique can also be used to test speculative or implied links between situations, such as scenes and/or samples there from, based on other information or evidence.

As well as applying the technique in this way it is possible to use a limited number of test results from a plurality of situations to produce a list of supported genotypes given those test results. These likely genotypes can then form the basis of further searches or investigations. A given supported genotype can be compared with records of genotypes determined or suggested for other situations, such as from other samples, for other scenes or from the testing of individuals to determine their genotype. A match gives a link between them. The search can include not only direct matches but also take into account the fact that not all the prior genotypes may be complete. A match, therefore, may be deemed to extend to genotypes where the two are close to one another, for instance where they share 20 or 21 out of the 22 alleles considered in the determination of a test result. Again the genotype links suggested can link a variety of situations, such as scenes and/or samples and/or individuals and/or events in a useful and informative way.

1. A method of considering DNA based links between two or more situations, the method including:
obtaining a plurality of test results, each test result relating to a situation, each test result including information on the DNA from that situation, the plurality of test results providing a group of test results;
selecting a plurality of test results from the group of test results, the plurality of results forming a combination of test results;
considering a genotype as possibly giving rise to each of the test results of the combination, evaluating the support for that genotype giving rise to all of the test results of the combination;
considering the genotype as a DNA based link between the situations for the test results in the combination if the support meets defined criteria.

2. A method according to claim 1 in which the method includes the further step of comparing a genotype which is considered a DNA based link against records of genotypes to identify matching genotypes in the records.

3. A method according to claim 1 in which the evaluation involves a direct evaluation of the support for a genotype giving the combination of test results or involves an evaluation of the support for a given genotype giving each of the test results, the individual evaluations being combined to give the overall evaluation.

4. A method according to claim 1 in which the support meets the defined criteria when the probability that the genotype could have given rise to the test results of the combination is above a given level and/or when an expression of the support that the genotype could have given rise to the test results of the combination is below a given level.

5. A method according to claim 1 in which the evaluation of the support for a genotype giving rise to the combination of test results includes a consideration of the probability of the test results arising given that genotype and the probability of occurrence of that genotype.

6. A method according to claim 1 in which the evaluation of the support for a genotype giving rise to the combination of test results is defined by
$$Pr ( Gr \vert D… ) = \frac{{\prod }_{\text{?}} Pr ( D \text{?} \vert Gr )}{{\sum }_{i} {\prod }_{\text{?}} Pr ( D \text{?} \vert Gr ) Pr ( Gr )} for all 1. \\ \text{?} \text{indicates text missing or illegible when filed}$$
where Gₗ represents the particular genotype, D represents the combination of test results, potentially including test results due to various scenes and/or samples from scenes and/or replicates of samples from scenes, i represents the range of replicates, j the range of samples, k the range of scenes and l the range of genotypes under consideration.

7. A method according to claim 1 in which one or more limits are applied to the genotypes which are considered from amongst the full set of possible genotypes, the limits being based on one or more rules as to genotypes which could not practically give one or more of the results in the combination being considered.

8. A method according to claim 1 in which the evaluation of the support for a genotype giving rise to the group of test results includes a consideration of the effect of one or more of contamination of the test results and/or allele drop out from the results and/or stutter in the results and/or preferential amplification of the results.

9. A method according to claim 1 in which a genotype which is considered as a DNA based link between the situations of the combination is used in a further consideration, the further consideration including the review of possible matches between the genotype and a collection of genotype records.

10. A method of considering DNA based links between two or more situations, preferably according to claim 1, the method including:
obtaining a first test result for a first situation, the first test result including information on the DNA from the first situation;
considering a genotype as possibly giving rise to the first test result and evaluating the support that the genotype gave rise to the first test result, repeating the evaluation of the support for a plurality of other genotypes, generating a set of possible genotypes based on the evaluation with respect to the first test result;
obtaining a second test result for a second situation, the second test result including information on the DNA from the second situation;
considering a genotype as possibly giving rise to the second test result and evaluating the support that this genotype gave rise to the second test result, repeating the evaluation of the support for a plurality of other genotypes, generating a set of possible genotypes based on the evaluation with respect to the second test result;
combining the set of possible genotypes for the first test result and the set of possible genotypes for the second test result, genotypes present in the first set and the second set being given a higher ranking in the combined set than genotypes not present in one or more of the sets;
considering one or more of the higher ranked genotypes in the combined set as a DNA based link between the first situation and the second situation.

11. A method according to claim 10 in which the evaluation of the support involves a determination of the mixture proportions contributed by different individuals.

12. A method according to claim 10 in which the evaluation of the support is used to rank the set.

13. A method according to claim 10 in which the evaluation produces a list of possible genotypes for the first and second test results.

14. A method according to claim 10 in which the combining of the set for the first test result and the set for the second test result includes, for genotypes present in the first set and in the second set, adding the support for that genotype for the first set to the support for that genotype for the second set.

15. A method according to claim 10 in which the combining of the set or ranked evaluation for the first test result and the set or ranked evaluation for the second test result includes combining the support for that genotype for the set or ranked evaluation it is present in with a dummy support for the set or ranked evaluation it is absent from.

16. A method according to claim 10 in which the genotypes are ranked within the combined set or combined evaluation, genotypes present in each of the sets or ranked evaluations receiving a high ranking in the combined set or ranked evaluation and/or genotypes absent from one or more of the sets of ranked evaluations receiving a low ranking in the combined set or ranked evaluation.
